(19) United States
(12) Patent Application Publication
(10) Pub. No.: US 2007/0092589 A1
(43) Pub. Date: Apr. 26, 2007

(54) VETERINARY PHARMACEUTICAL COMPOSITIONS FOR GASTRIC ULCERS

(75) Inventor: Ralph Stanley Robinson, Guelph (CA)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: SELECTED BIOPRODUCTS INC., Guelph (CA)

(21) Appl. No.: 11/552,840

(22) Filed: Oct. 25, 2006

(30) Foreign Application Priority Data
Oct. 25, 2005 (CA) 2,524,425

(51) Int. Cl. A61K 36/48; A61K 36/28

(52) U.S. Cl. 424/757; 424/764; 424/769; 424/773

(57) ABSTRACT

A veterinary polyherbal pharmaceutical composition for treatment of gastric ulcers in horses, the composition comprising the following ingredients in the following weight ranges:

| | Weight Range |
|---|---|
| Composition | (% by weight) |
| Deglycerinized Licorice Root | 30-50 |
| Meadowsweet Herb | 10-20 |
| Marshmallow Root | 10-15 |
| Slippery Elm Root | 0-5 |
| Chamomile | 15-20 |
| Yarrow Leaf | 0-5 |
| Fenugreek Seed | 0-5 |
| Milk Thistle | 5-10 |

### FIELD OF THE INVENTION

The present invention relates to a veterinary polyherbal pharmaceutical composition for treatment of gastric ulcers in horses.

### BACKGROUND OF THE INVENTION

Prior to the present invention, gastric ulcers in horses have been alleviated by the use of veterinary pharmaceuticals. Such drugs produce numerous side effects, not the least of which is compromised digestion due to the suppression of stomach acid.

### SUMMARY OF THE INVENTION

The present invention relates to a completely natural product for treatment of gastric ulcers in horses, the product having a composition as follows:

| | Weight Range (% by weight) |
|---|---|
| Deglycerinized Licorice Root | 30-50 |
| Meadowsweet Herb | 10-20 |
| Marshmallow Root | 10-15 |
| Slippery Elm Root | 0-5 |
| Chamomile | 15-20 |
| Yarrow Leaf | 0-5 |
| Fenugreek Seed | 0-5 |
| Milk Thistle | 5-10 |

In a specific form of the polyherbal blend according to the present invention, the ingredients are present in the following percentages by weight:

| | Weight Range |
|---|---|
| Composition | (% by weight) |
| Deglycerinized Licorice Root | 40 |
| Meadowsweet Herb | 15 |
| Marshmallow Root | 12.5 |
| Slippery Elm Root | 2.5 |
| Chamomile | 17.5 |
| Yarrow Leaf | 2.5 |
| Fenugreek Seed | 2.5 |
| Milk Thistle | 7.5 |

The product according to the present invention is completely natural and stimulates appetite in horses within 48 hours of application, healing ulcers in 21 to 30 days in test animals undergoing a full training and race schedule.

### EXAMPLE

24 animals at 2 locations were endoscoped by Dr. Alison Moore Campbellville, Ontario; following which they were dosed with a polyherbal pharmaceutical composition comprising:

| | Weight Range |
|---|---|
| Composition | (% by weight) |
| Deglycerinized Licorice Root | 40 |
| Meadowsweet Herb | 15 |
| Marshmallow Root | 12.5 |
| Slippery Elm Root | 2.5 |
| Chamomile | 17.5 |
| Yarrow Leaf | 2.5 |
| Fenugreek Seed | 2.5 |
| Milk Thistle | 7.5 |

being fed 75 ml twice daily over a 30 day period. This treatment was followed by GI endoscopy by the same veterinarian. Otherwise, the test subjects followed a normal feeding, training/racing regime.

They were examined initially, before treatment and after the 30 days of treatment. Over this period of time, all animals had a minimum 1 grade reduction of ulceration; many had 2 or 3 grade improvements. There was complete healing evidence in some animals. Trainers reported improved feed consumption and improved race times as well as attitudes, the latter possibly due to pain reduction.

Treatment of horses with the composition according to the present invention results in lower costs than drug therapy and, because conventional drugs are not used, results in no withdrawal in the horses following treatment and, for race horses, no positive testing for drugs during the treatment.

While the invention has been described in conjunction with illustrated embodiments, it will be understood that it is not intended to limit the invention to such embodiments. On the contrary, it is intended to cover all alternatives, modifications and equivalents as may be included within the spirit and scope of the invention as defined by the present patent specification as a whole.

1. A veterinary polyherbal pharmaceutical composition for treatment of gastric ulcers in horses, the composition comprising the following ingredients in the following weight ranges:

| | Weight Range |
|---|---|
| Composition | (% by weight) |
| Deglycerinized Licorice Root | 30-50 |
| Meadowsweet Herb | 10-20 |
| Marshmallow Root | 10-15 |
| Slippery Elm Root | 0-5 |
| Chamomile | 15-20 |
| Yarrow Leaf | 0-5 |
| Fenugreek Seed | 0-5 |
| Milk Thistle | 5-10 |

2. A composition according to claim 1, wherein the ingredients are present in the following percentages by weight:

| | Weight Range |
|---|---|
| Composition | (% by weight) |
| Deglycerinized Licorice Root | 40 |
| Meadowsweet Herb | 15 |
| Marshmallow Root | 12.5 |
| Slippery Elm Root | 2.5 |
| Chamomile | 17.5 |
| Yarrow Leaf | 2.5 |
| Fenugreek Seed | 2.5 |
| Milk Thistle | 7.5 |
